(19) United States
(12) Patent Application Publication
(10) Pub. No.: US 2018/0116920 A1
(43) Pub. Date: May 3, 2018

(54) MULTI-STEP PROCESS FOR NO PRODUCTION

(71) Applicant: BSN medical GmbH, Hamburg (DE)

(72) Inventors: Karsten HEMMRICH, Meerbusch (DE); Annahit ARSHI, Hamburg (DE); Christian SCHULZE, Tostedt (DE)

(21) Appl. No.: 15/569,320

(22) PCT Filed: Apr. 26, 2016
(86) PCT No.: PCT/EP2016/059312

§ 371 Date: Oct. 25, 2017

(30) Foreign Application Priority Data
Apr. 29, 2015 (EP) 15165747.5

(51) Int. Cl. A61K 8/19; C01B 21/24; A61Q 19/00; A61K 8/67; A61K 8/49

(52) U.S. Cl. CPC A61K 8/19; C01B 21/24; A61Q 19/00; A61K 2800/522; A61K 8/498; A61K 2800/81; A61K 8/676

(57) ABSTRACT

The present invention relates to a multi-step process for the production of nitrogen monoxide (NO) from a pH-labile NO donor in the presence of at least one antioxidant. The invention also relates to a device for implementation of said process and the use of said device for the treatment of diseases. Moreover, the invention relates to a cosmetic process using said process or said device.

### SUBJECT MATTER OF THE INVENTION

The present invention relates to a multi-step process and a corresponding device for the production of nitrogen monoxide (NO). The invention also relates to the utilisation of said process and device for the treatment of diseases, in particular of diabetes-related circulatory disorders and lower limb wounds.

### BACKGROUND OF THE INVENTION

Numerous processes and devices for the production of NO are known in the prior art.

According to EP 1 903 003 A1, NO can be produced by photolysis of a photo-labile NO precursor, whereby the reaction proceeds in the presence of radical scavengers and antioxidants and leads to the production of highly pure NO. Applying this process to the production of NO in liquids, usually only a slow increase of the NO concentration is to be expected.

According to WO2013/063354, an NO-releasing foot bath can be produced by adding a polysiloxane polymer derivatised with diazeniumdiolate groups to the bath solution. This polymer then reacts with water to produce NO. Since NO is generated in this context through spontaneous disintegration of the polymer side groups, the release kinetics can be controlled only to an insufficient degree. Moreover, it takes a significant period of time for a therapeutically relevant NO level to be established in this process.

Therefore, there continues to be a need for new processes for the production of NO-containing solutions, in which NO can be produced rapidly and at high purity, yet in controlled manner.

It is therefore the object of the invention to provide a process for the production of NO that is improved with respect to at least one of the drawbacks specified above.

### SUMMARY OF THE INVENTION

Said object is met according to the invention, in that a process for the production of nitrogen monoxide (NO) that comprises the following steps is provided:
(a) Providing a carrier medium comprising at least one pH-labile NO donor;
(b) adjusting the pH value of the carrier medium to a pH value that induces the decomposition of the at least one pH-labile NO donor while producing NO;
(c) maintaining a pH value that induces the production of NO for a period of time that permits the production of a physiologically relevant amount of NO;
(d) increasing the pH value of the carrier medium;
(e) optionally, adding another at least one additional antioxidant in step (d) or in a subsequent step (e);
   whereby the carrier medium contains, in addition, at least one antioxidant in step (a) or the at least one antioxidant is added in step (b).

Specific embodiments of the invention are the subject matter of further dependent and independent claims.

The process according to the invention combines several decisive advantages as compared to the processes known from the prior art.

It has been evident, surprisingly, that this process meets the complementary requirements of a NO release kinetics. Accordingly, a therapeutically relevant concentration of NO in the carrier medium can be built up very rapidly in the acidic medium, and can then be maintained over an extended period of time in controlled manner after increasing the pH.

Usually, the short half-life of NO renders its therapeutic use difficult. The process according to the invention allows the NO level to be maintained for a sufficient period of time, despite the short half-life, due to a stabilisation of the NO in the neutral or alkaline carrier medium.

Due to the presence of antioxidants, the process allows NO to be produced at appropriate purity as required for therapeutic or cosmetic application.

Numerous pH-labile and photo-labile NO donors are known from the prior art, such as, for example, nitrite salts, NONOates or nitrosothiols, which a person skilled in the art can use in this context.

Due to the high level of control over the release, the process can be used in devices that release only very small amounts of NO. This is a decisive benefit especially with NO since it is a highly potent bioactive molecule. Moreover, this permits the development of a corresponding device (such as, for example, a wound dressing or a foot bath) as a medical device (e.g. as a so-called class III medical device) in as far as this concerns a device, in which the effect is caused primarily by the mechanical or physical properties of the device.

Simple adaptation of the process with respect to NO donors, acids and irradiation sources allows the process to be adapted specifically to the requirements of the treatment.

With the process according to the invention, there is no need for an external supply of NO.

The process according to the invention is a simple process that uses mainly known substances and is not only inexpensive and can be implemented in a non-complex way and manner, but it is also easy to use in therapeutic applications and has a low error rate.

Devices that are operated with said production process afford more freedom with respect to the characteristic parameters and the selection of materials.

In summary, the NO production process according to the invention enables NO-based forms of therapy, in which the highly reactive, but correspondingly unstable, gas NO can be applied in controlled manner, inexpensively, reliably, safely and in a manner that can be individualised by the user.

### DETAILS OF THE INVENTION

Accordingly, the invention includes a two-step process, in which the generation of NO is induced in an acidic medium initially and, after a selected period of time, the pH value is increased in order to stop or reduce the pH-dependent synthesis of new NO and to provide an NO-containing carrier medium.

Due to the pH being increased to the preferred neutral or alkaline range, the new generation of toxic NO₂ radicals is omitted. Due to the inventive presence of the at least one antioxidant, NO₂ radicals and other radicals arising during the generation of NO are eliminated such that the carrier medium is enriched in highly pure NO.

The process starts with a carrier medium that comprises at least one pH-labile NO donor.

Furthermore, the carrier medium must contain at least one antioxidant at the point in time, at which an acidic pH value permits the generation of NO. For this purpose, the antioxidant can already be present in the carrier medium in step (a). This is advantageous in that the ingredients contained in the carrier medium are protected by the at least one antioxidant contained therein from undesirable oxidation as early as during the production and/or storage. This can be beneficial especially with inventive devices such as wound dressings or plasters, since the addition of other substances is difficult in this case and since these need to be sufficiently stable during storage.

Alternatively, the at least one antioxidant can be added in step (b). This makes sense, especially, if the antioxidant disadvantageously interacts with the carrier medium or an ingredient contained therein or itself is unstable in the carrier medium. Moreover, this allows an antioxidant to be used which simultaneously, acting as an acid, induces the generation of NO. Pertinent examples are ascorbic acid or uric acid.

Carrier Medium

Any medium that is capable of taking up and releasing NO can be used as carrier medium in this context. Preferably, the carrier medium is selected from the group containing foam, gel, cream, and liquid. Preferably, the carrier medium is a liquid and, in particular, an aqueous liquid.

Said carrier medium can be present as a separate phase, but it can also be embedded in a substrate. Accordingly, an aqueous liquid can be present as a bath solution, but it can also be contained in a layer of a wound dressing, for example after having been absorbed by a liquid-absorbing matrix.

NO Donor

pH-labile NO precursors (NO donors, NOD) are known in the prior art and are known to a person skilled in the art.

In a preferred embodiment of the invention, the pH-labile NO donors are selected from the group containing organic nitrates, inorganic nitrates, inorganic nitrites, organic nitrite esters such as alkyl nitrites, sulfur-, nitrogen- or oxygen-nitroso compounds, NO-metal compounds, and NO-chelating substances.

Examples of pH-labile NOD comprise inorganic nitrites, alkylnitrites such as isopentylnitrite, diazeniumdiolates (e.g. U.S. Pat. No. 7,105,502; 7,122,529; 6,673,338), trans[RuCl([15]aneN4)NO]²⁺, nitrosyl ligands, 6-nitrobenzo[a]pyrrole, S-nitroso-glutathione, S-nitroso-thiols, S-nitroso-N-acetyl-D-penicillamine (SNAP), nitroaniline derivatives (see US 2013/0224083 A1), 2-methyl-2-nitrosopropane, imidazolyl derivatives, nitrate esters, hydroxylnitrosamine, hydroxylamine, hydroxyurea or sodium nitroprusside.

Preferably, the pH-labile NO donor is an inorganic nitrite salt, which expediently is a pharmacologically tolerable substance. For example, nitrites of alkali or alkaline earth metals are used as such. To name some examples: LiNO₂, NaNO₂, KNO₂, RbNO₂, CsNO₂, FrNO₂, Be(NO₂)₂, Mg(NO₂)₂, Ca(NO₂)₂, Sr(NO₂)₂, Ba(NO₂)₂ or Ra(NO₂)₂ and combinations thereof.

NaNO₂ is particularly preferred as NOD and can be used, in further preferred manner, together with a combination of ascorbic acid and Trolox as antioxidants in the carrier medium.

In this context, the concentration of the nitrite salts, relative to the total weight of the carrier medium containing them, can be up to 20% by weight, preferably between 0.25 and 10% by weight, particularly preferably between 3 and 7.5% by weight.

In an alternative embodiment, a nitrate salt that is subject to enzymatic conversion to the corresponding nitrite salt can be used just as well. In this context, nitrates of alkali or alkaline earth metals are preferably used as such. To name some examples: LiNO₃, NaNO₃, KNOB, RbNO₃, CsNO₃, FrNO₃, Be(NO₂)₃, Mg(NO₂)₃, Ca(NO₂)₃, Sr(NO₂)₃, Ba(NO₂)₃ or Ra(NO₂)₃. In this context, the concentration of the nitrate salts, relative to the total weight of the carrier medium containing them, can be up to 20% by weight, preferably between 0.25 and 10% by weight, particularly preferably between 3 and 7.5% by weight.

Antioxidant

To be able to remove the multiply oxidised nitrogen oxides, oxygen radical anions or hydroxyl radicals that arise during the generation of NO, the carrier medium needs to comprise at least one antioxidant.

Based on their chemical mode of action, antioxidants are sub-classified as radical scavengers or reducing agents.

Often, chain reaction-like radical transfers take place during oxidation reactions involving organic compounds. In this context, substances with sterically hindered phenol groups take effect and produce inert, stable radicals in the course of these transfers, which do not keep on reacting such that the reaction cascade is terminated (radical scavenger). These include natural substances such as the tocopherols and synthetic substances such as butylhydroxyanisol (BHA), butylhydroxytoluene (BHT), and gallates. They are to be used, in particular, with non-polar carrier media.

Moreover, reducing agents with a very low standard redox potential of less than +0.4 V (at pH 7.0 and 25° C.) can be used as well. Typical representatives include ascorbic acid (−0.04 V at pH 7 and 25° C.), sulfurous acid salts (+0.12 V at pH 7 and 25° C.), and certain organic sulfur-containing compounds (e.g. glutathione, cysteine, thiolactic acid), that can be used predominantly in hydrophilic carrier media.

In a preferred embodiment, the at least one antioxidant is capable of reducing HNO₂, which is present as NO donor in acidic medium, to NO. For this purpose, the antioxidant, as a reducing agent, has to have a standard redox potential of less than +1.0362 Volt, preferably of less than +0.5 Volt, particularly preferably of less than +0.2 Volt, and even more particularly preferably of less than 0 Volt.

Expediently, the at least one antioxidant is capable of reducing the harmful NO₂ radical to the NO₂⁻ anion. For effective elimination of the NO₂ radical, the bimolecular reaction constant k of the at least one antioxidant should preferably be more than 1.0×10⁶ M⁻¹s⁻¹ and preferably be more than 1.0×10⁷ M⁻¹s⁻¹. Antioxidants that are suitable according to the invention as well as the corresponding reaction constants are disclosed in Kirsch et al., 2002 (Biol. Chem 383; 389-399, see Table 1). To name some examples: Captopril thiolate, caffeic acid, sinapinic acid, ferulic acid, lycopene, zeaxanthin, lutein, astaxanthin, canthaxanthin, arachidonate, Gly-Tyr dipeptide, tyrosine, purines and pyrimidines such as the nucleobases adenine, guanine, cytosine, thymine, uracil, and the corresponding derivatives and analogues thereof including the nucleosides and nucleotides containing them.

In a further embodiment, the carrier medium used according to the invention, which preferably is a liquid, contains not only the antioxidant, but an anti-oxidation synergist as well. Synergists support the effect of antioxidants, for example by regenerating spent antioxidants (so-called “redox cycling”). By complexing traces of metals (sodium EDTA) or by producing an oxidation-inhibiting pH value, synergists can reinforce the antioxidative effect of a radical scavenger or reducing agent. Typical examples of anti-oxidation synergists include EDTA, 1-hydroxyethane-1,1-diphosphonic acid, citric acid, fumaric acid, uric acid, and 2-(hydroxymethyl)-1,4-benzyldiol.

It is particularly preferable in the production process according to the invention to use ascorbate or ascorbic acid as the antioxidant.

A person skilled in the art is aware of numerous antioxidants that are capable of degrading or neutralising multiply-oxidised nitrogen oxides, oxygen radical anions, hydroxyl radicals, or aqua-complexed electrons. A person skilled in the art will select these according to the respective composition of the carrier medium.

Antioxidants such as, for example, tocopherols, tocotrienols, tocomonoenols, Irganox®, Irgafos®, butylhydroxyanisol (BHA) and butylhydroxytoluene (BHT) are well-suited for apolar carrier media, such as, for example, apolar solvents, creams or gels.

Water-soluble vitamin E derivatives such as Trolox or alpha-AMG, organic sulfur-containing compounds such as glutathione, cysteine or thiolactic acid or organic acids such as ascorbic acid, alpha-lipoic acid, hydroxycinnamic acids such as p-cumaric acid, ferulic acid, sinapinic acid or caffeic acid, or hydroxybenzoic acids such as gallic acid, protocatechuic acid, syringic acid or vanillic acid are well-suited for polar media such as, for example, aqueous liquids or hydrogels.

Other preferred antioxidants comprise polyphenolic compounds such as anthocyanins, flavonoids and phytooestrogens.

In a preferred embodiment, the at least one antioxidant from step (a) or (b) is a mixture of one representative of the reductone group and one representative of the 6-hydroxy-chroman group or of the thiols. It has been evident, according to the invention, which said combination of antioxidants is particularly effective in eliminating the harmful radicals that are generated during the reaction without adversely affecting the production of NO.

In a preferred embodiment, a combination of antioxidants according to the following table is used in step (a) or (b). The advantageous use of these combinations is based on the fact that a first antioxidant preferably reduces the HNO₂ (antioxidant I) and a second antioxidant preferably traps the harmful NO₂ radical (antioxidant II). The table shows, on the one hand, the general substance class, and then discloses, in exemplary manner, some preferred concise combinations of substances.

| Antioxidant I | Antioxidant II |
|---|---|
| Reductone | 6-Hydroxychroman |
| Ascorbic acid | Trolox |
| Isoascorbic acid | Trolox |
| Erythroascorbic acid | Trolox |
| Ascorbyl stearate | alpha-Tocopherol |
| Ascorbyl palmitate | alpha-Tocopherol |
| Reductone | Thiol |
| Ascorbic acid | Cysteine |
| Isoascorbic acid | Cysteine |
| Erythroascorbic acid | Cysteine |
| Ascorbyl stearate | Cysteine |
| Ascorbyl palmitate | Cysteine |
| Ascorbic acid | Glutathione |
| Isoascorbic acid | Glutathione |
| Erythroascorbic acid | Glutathione |
| Ascorbyl stearate | Glutathione |
| Ascorbyl palmitate | Glutathione |

According to the invention, a representative of the reductone group shall be understood to be an organic chemical compound that bears two hydroxyl groups on the two carbon atoms of a C═C double bond (“endiol”) and, in addition, a carbonyl group right on the neighbouring carbon atom. The double bond of these endiols is stabilised due to the conjugation with the carbonyl group such that mainly the endiol form, rather than the keto form, is present in the tautomeric equilibrium (“keto-enol tautomerism”). Reductones, being vinylogous carboxylic acids, show an acidic reaction. The reductone group comprises, for example, ascorbate and derivatives thereof, hydroxypropandial (tartronaldehyde), trans-3,4-dihydroxy-3-hexen-2,5-dione (DHHD), and 2,3-dihydroxy-2-cyclopentenone (reductinic acid). Preferably, ascorbic acid or ascorbate and derivatives thereof, such as erythroascorbic acid or ascorbyl palmitate, are used as the representative of the reductone group.

According to the invention, representatives of the 6-hydroxychroman group are substances that comprise a chroman ring that is hydroxylated in 6-position and, in addition, can bear one or further substituents (preferably methyl) rather than hydrogen at the other positions. Typical representatives of the 6-hydroxychromane group include the tocopherols. Tocomonoenoles, and tocotrienoles and derivatives thereof such as, for example (RS)-6-hydroxy-2,5,7,8-tetramethylchroman-2-carboxylic acid (Trolox). Preferably, alpha-tocopherol or Trolox is used as representative of the 6-hydroxychroman.

According to the invention, thiols (also called thio alcohols), are organic chemical compounds that bear as functional groups one or more thiols (—SH) through an aliphatic or aromatic bond. Cysteine and glutathione are preferred as thiols.

In this context, the final concentration of the thiols in the carrier medium preferably is between 1 and 1,000 mM, particularly preferably between 20 and 200 mM, and even more particularly preferably between 50 and 100 mM.

For a polar carrier medium, such as, for example, an aqueous liquid or a hydrogel, it is expedient to combine water-soluble representatives of the group specified above, for example ascorbate and (RS)-6-hydroxy-2,5,7,8-tetramethylchroman-2-carboxylic acid(Trolox), ascorbate, and cysteine, or, preferably, ascorbate and N-acetylcysteine.

For a non-polar carrier medium, such as, for example, a cream based on oil or fat, it is expedient to use two lipophilic representatives of the group specified above, for example, ascorbyl palmitate, ascorbyl stearate and alpha-tocopherol, and preferably a combination of ascorbyl palmitate and alpha-tocopherol or ascorbyl stearate and alpha-tocopherol.

Expediently, the at least one antioxidant is present at a molar excess over the NO donor.

Combining two antioxidants reacting preferably with HNO₂ and NO₂ radical (referred to as antioxidant I and antioxidant II in the scope of the invention), it is advantageous that these are present at a molar ratio according to the following formula:

mol [NO donor]<mol [antioxidant I]<mol [antioxidant II].

Since the elimination of NO₂ radicals is a particularly important task especially for therapeutic and cosmetic applications, antioxidant II should be present at a higher molar ratio for safety reasons.

Preferably, the carrier medium contains the following three components in step (a) or (b): NO donor, antioxidant I, and antioxidant II at a molar ratio of 1:2-20:4-100, whereby the molar ratio: is nitrite<ascorbate<Trolox. A preferred molar ratio in this context is 1:2-10:5-50, particularly preferably 1:3-8:5-20, and specifically a ratio of 1:5:10.

In one embodiment of the invention, and in particular in its embodiment as an aqueous liquid, the carrier medium additionally contains one or more of the following substances: Catalysts, detergents, buffer substances, chromophores, substances stabilising the prodrug such as, for example, dimethyl sulfoxide or ethanol, substances increasing the half-life of NO such as are disclosed in US 2003/0039697, NOD stabilisers, antioxidants, dyes, pH indicators, care agents, fragrances, pharmacological agents.

A person skilled in the art will select suitable substances or mixtures of substances with a view to the respective purpose and based on the person's general professional knowledge. The person skilled in the art will specifically make sure that physiologically compatible and/or dermatologically compatible substances or mixtures of substances are used if the carrier medium is to be used for topical application.

Acidic Activation in Step (b)

The liquid is made to have an acidic pH value for cleavage of the pH-labile NO donor. According to the invention, said pH value is sufficiently low such that it induces cleavage of the pH-labile NO donor while producing NO. The actual pH value depends on the pH-lability of the NO donor and the desired period of time for generation of NO. The lower the pH value, the faster the NO will be generated in the carrier medium.

According to the invention, the pH value in step (b) is between 0.0 and 6.9, preferably between 2.0 and 6.0, particularly preferably between 4.5 and 6.0, and in particular is 5.0. As mentioned above, the optimal pH value depends on the specific NO donor that is used and the intended reaction rate and can be adjusted appropriately by a person skilled in the art.

In an embodiment of the invention, the acidic medium required in the process for release of NO from the pH-labile NO donor is generated through the addition of an acid or of a buffer with an acidic pH value (i.e. with a pH<7).

Numerous acids are available for use as acids for this purpose by a person skilled in the art. These comprise not only mineral acids such as HCl, H₂SO₄, H₃PO₄ or HNO₃, but also organic acids such as acetic acid, citric acid or lactic acid.

In a particular embodiment, the acid is an antioxidant as well, such as, for example, ascorbic acid or thiolactic acid, or an anti-oxidation synergist such as 1-hydroxyethan-1,1-diphosphonic acid or uric acid. By this means, no antioxidant needs to be present in step (a). The antioxidant is added in the form of an acid in step (b) and thus gets to be effective specifically at the point in time from which harmful or undesired radicals arise as a result of the NO donor being cleaved.

In another embodiment, the acid is present as a solid in the solid carrier medium and is dissolved, and thus can become deprotonatable, upon the addition of water. In this context, the acid can be present in the form of a powder, granulate, nanoparticles or acid groups situated on a polymer.

Photo-Latent Acid

In a preferred embodiment of the invention, the generation of NO in step (b) is initiated through the activation of a photo-latent acid, which releases the acid, i.e. acidifies the liquid, as a consequence of being irradiated with the electromagnetic radiation. This is advantageous in that no acid from outside needs to be added to the reaction, but that, rather, the acidification can be induced by a substance that is present in the carrier medium anyway.

This embodiment is particularly advantageous if additional NO is generated in step (e) through a photolytic process, since this means that the light source is already provided in the process according to the invention and the corresponding device.

Moreover, it is advantageous in this context that the irradiation, as the initial event, can induce a longer-lasting release of NO and thus acts like a “switch” that starts the generation of NO according to the invention.

Examples of photo-latent acids include, e.g., onium salts, such as sulfonium or iodonium salts, as well as oxime sulfonic acid esters. Said compounds are known in the art and are described in numerous literature references.

Examples include triarylsulfonium or diaryliodonium salts, e.g. not substituted or substituted with alkyl or alkoxy substituents with a wide variety of anions such as, for example, HSO₄⁻, PF₆⁻, SbF₆⁻, AsF₆⁻, Cl⁻, Br⁻, I⁻, ClO₄⁻, PO₄⁻, SO₃CF₃⁻, tosylate, or a borate anion, such as BF₄⁻ or B(C₆F₅)₄⁻.

Onium salts have been described, e.g., by J. V. Crivello, K. Dietliker “Photoinitiators for Free Radical, Cationic & Anionic Photopolymerisation”, Volume III of “Chemistry & Technology of UV & EB Formulation for Coatings, Inks & Paints”, 2nd Ed., J. Wiley and Sons/SITA Technology (London), 1998 (specifically pages 464-466). lodonium salts are known from a large number of patent publications, e.g. U.S. Pat. No. 4,151,175, U.S. Pat. No. 3,862,333, U.S. Pat. No. 4,694,029, EP 562897, U.S. Pat. No. 4,399,071, WO 98/46647, etc., and are, e.g., “symmetrical” or “non-symmetrical” diaryliodonium compounds of formula (C)

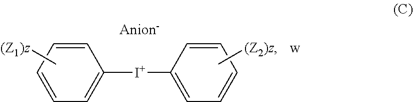

whereby Z₁ and Z₂ are identical or different and are, for example, linear or branched C₁-C₂₀ alkyl, C₁-C₂₀ alkoxy, halogen, C₂-C₁₂ alkenyl, cycloalkyl; and z, independent of each other, are 0 to 5, in particular are 0 or 1, i.e. in case multiple residues Z₁ or Z₂ are present, i.e. z is more than 0, all Z₁ or all Z₂ do not have to have the same meaning.

Additional photo-latent acid donors have been summarised by M. Shirai and M. Tsunooka in Prog. Polym. Sci., Vol. 21, 1-45 (1996), in the form of an overview.

Other suitable photo-latent acids include the oxime sulfonates. Said compounds are also known in the art and have been disclosed, for example, in U.S. Pat. No. 5,237,059, EP 571330, EP 241423, EP 139609, EP 361907, EP 199672, EP 48615, EP 12158, and EP 780729.

Examples include α-(methylsulfonyloxyimino)-4-methoxybenzylcyanide, α-(methylsulfonyloxyimino)-3-methoxybenzylcyanide, α-(methylsulfonyloxyimino)-3,4-dimethylbenzylcyanide, α-(methylsulfonyloxyimino)-thiophen-3-acetonitrile, α-(isopropylsulfonyloxyimino)-thiophen-2-acetonitrile, cis/trans-α-(dodecylsulfonyloxy-imino)-thiophen-2-acetonitrile, ESACURE (Lamberti), IRGACURE (Ciba) e.g. IRGACURE® PAG103 (2-methyl-α-[2-[[[(n-propyl)sulfonyl]oxy]imino]-3(2H)-thienylidenFbenzylaceto-nitrile, 2(5H)-thienyliden]-Benzylacetonitrile), IRGACURE® PAG108 (2-methyl-α-[2-[[[(n-octyl)sulfonyl]oxy]imino]-3(2H)-thienyliden]-benzyl-acetonitrile), IRGACURE® PAG121 (2-methyl-α-[2-[[[(4-methylphenyl)sulfonyl]oxy]imino]-3(2H)-thienyliden]-benzylacetonitrile), IRGACURE® PAG203, ethanone, 1,1′-[1,3-propandiylbis(oxy-4,1-phenylen)]bis-[2,2,2-trifluoro-bis[O-(propylsulfonyl) oxime], UVI (DOW Chemicals), CYRACURE (DOW Chemicals), and 2-(-methoxystyryl)-4,6-bis(trichloro-methyl)-1,3,5-triazine (Sigma Aldrich).

The oxime sulfonates described in WO 2000/1097 A2 or GB 2348644 are also well-suited. Oxime compounds that release acids other than sulfonic acids are also well-suited and are disclosed, for example, in WO 00/26219.

The listing above shall be understood in the context of the present invention as being exemplary only and in no way as being limiting.

According to the invention, photo-latent Lewis acids are preferred in this context. The photo-latent Lewis acid is a photo-chemically active substance. I.e. a substance that is capable of taking up energy from incident light in appropriate manner such that the substance, due to the uptake of energy, is changed in a chemical reaction and releases a free Lewis acid in the process.

For this purpose, the photo-latent Lewis acid has an absorption different from zero at the wavelengths of the incident light, whose dose needs to be monitored in the respective case, such that the radiation is absorbed completely or at least partly by the photo-latent Lewis acid and transfers it to an energetically excited state. The energetically excited state results in the release of the Lewis acid. By this means, the concentration of free Lewis acid in the carrier medium is increased locally, which results in acid-induced cleavage of the pH-labile NO donor.

Basically, any substance is conceivable as a photo-latent Lewis acid if it comprises an absorption different from zero at least in a wavelength range of the radiation and moreover is capable of releasing a Lewis acid as a result of absorbing the radiation, i.e. to generate it in a chemical reaction or otherwise provide it as a free compound, for example in a desorption step or from a Lewis adduct. The Lewis acid can, for example, be a part that is cleaved off the photo-latent Lewis acid.

All electrophilic electron pair acceptors are understood to be Lewis acids, i.e. all substances that can take up electron pairs, for example molecules and ions with an incomplete noble gas configuration, i.e. an electron gap.

In particular, in the scope of the present invention, Bronsted acids (classical acids; protonic acids) are considered Lewis acids, i.e. substances that are or contain proton donors, whereby this shall also include protons as such.

Examples of photo-latent Lewis acids that can be used according to the invention are known, for example, from WO 02/101462 A 1 and WO 2005/097876 A 1, to which reference shall be made expressly herewith.

According to WO 2005/097876 A 1, in particular Lewis acids based on a compound of general formula R¹-CH*R⁰-(A⁶)R²R³R⁴R⁵—OH are conceivable as latent Lewis acids. In this context, A⁶ is an aromatic ring system of six ring atoms, which can optionally contain one heteroatom or more heteroatoms and/or further annulated rings. R¹ is selected from the group comprising hydrogen, alkyl groups (in particular C₁-C₂₀ alkyl groups), alkenyl groups (in particular C₂-C₂₀ alkenyl groups), and aryl groups (in particular groups that are non-substituted or substituted by one, two or three C₁-C₄ alkyl groups, or C₁-C₄ alkoxy groups. R², R³, R⁴, and R⁵ are selected independent of each other from the group comprising hydrogen or functional substituents. R⁰ is selected from the group comprising C₁-C₆ alkyl groups or groups represented by general formula —Z¹-Q¹ or —Z²-Q². In this context, Z¹ is a single bond or a bridged sulfur atom (—S—) or oxygen atom (—O—) or a bridged secondary amine group (—NH—). In this context, Q¹ is a heterocyclic ring system with 5 to 9 ring atoms whose ring atoms can be carbon (C), sulfur (S), oxygen (O) and nitrogen (N), whereby the ring system contains at least two, preferably three, particularly preferably at least four carbon atoms. Specifically, Q¹ is morpholine, pyridine (possibly substituted with one to three C₁-C₂ alkyl groups or hydroxyl groups), mercaptobenzoxazole or mercaptobenzthiazole. Z² represents a C₁-C₄ alkylene group that can be substituted by one C₁-C₄ alkyl group or by Q³. Q² and Q³ independently represent phenyl groups that can be substituted, if applicable, by one to three C₁-C₄ alkyl groups, hydroxyl groups, C₅-₈ cycloalkyl groups and/or one heterocyclic ring system with 5 to 9 ring atoms, whose ring atoms can be carbon (C), sulphur (S), oxygen (O), and nitrogen (N), whereby the ring system contains at least two, preferably three, particularly preferably at least four carbon atoms. Moreover, the hydrogen atom H* that is bound to the carbon atom in alpha-position with respect to the substituent R″ can be cleaved off as a proton in a photochemical reaction upon the action of electromagnetic radiation.

Specific examples of photo-latent Lewis acids are described in WO 02/101462 A1, which can be used without exception without limitation by these examples.

The phenolic antioxidants described in WO 2003/050912 can also be used as photo-latent acids. Typical examples include compounds from the group of the hydroxyphenylbenzotriazoles, hydroxyphenyltriazines or hydroxybenzophenones, which all comprise a hydroxyl group arranged on a phenyl ring in ortho-position with respect to the bond between the phenyl ring and the main skeleton of the molecule.

In an embodiment of the invention, step (c), comprising the generation of NO, takes a period of time of between 5 seconds and 1 hour, preferably of between 1 and 30 minutes, particularly preferably of between 5 and 20 minutes, and even more particularly preferably of between 5 and 10 minutes.

The amount of NO generated in this context, for a solid carrier medium, is between 10 and 1,000 ppm, preferably between 100 and 750 ppm, and particularly preferably between 200 and 500 ppm.

Referring to liquid carrier media, it is preferable to express the concentration of the NO thus generated as the molar concentration and the molar concentration is between 0.01 and 2 mM, preferably between 0.05 to 1 mM, and particularly preferably between 0.1 and 0.5 mM.

Increasing the pH

The pH of the liquid is being increased in a process step (d) that follows after the primary generation of NO according to step (c). According to the invention, said increase in pH can take place by adding a base, an alkaline buffer system or by photoactivation of a photo-latent base.

According to the invention, the increase in pH according to the invention comprises one or more of the following features:
(a) increase of the pH value to pH 7.0 or more;
(b) increase of the pH value by at least one full pH increment;
(c) increase of the pH value to a pH value that is associated with a reduced generation of NO such that the amount of newly generated NO is equivalent to the decreasing amount of NO in the carrier medium.

According to the invention, the pH value is being increased sufficiently such that the acid-induced generation of NO is inhibited strongly or is fully prevented or such that, in a specific embodiment, the generation of NO is permitted at a reduced level such that the newly generated NO compensates for the decreasing concentration of NO (regardless of whether this is due to decomposition, reaction or release). In this sense, the increase in pH provides for the maintenance of a steady-state of NO.

Expediently, the increase in pH in step (d) in this context leads to a pH value that is between 4.0 and 12.0, preferably between 5.0 and 8.0, particularly preferably between 5.5 and 7.5, and in particular between 6 and 7.

Numerous bases are available as bases for use for this purpose by a person skilled in the art. These comprise both inorganic bases, such as NH₄OH, and organic bases, such as aliphatic or aromatic amines.

In one embodiment, a base is used for increasing the pH that is selected from the group containing NaOH, KOH, Ca(OH)₂, NH₄OH, and sodiumhydrogen carbonate.

In an alternative embodiment, an alkaline buffer is used for increasing the pH that is selected from the group containing phosphate buffer, barbital-acetate buffer, 4-(2-hydroxyethyl)-1-piperazinethanesulfonic acid (HEPES) buffer, tris(hydroxymethyl)-aminomethane (TRIS) buffer, 4-(2-hydroxyethyl)-piperazin-1-propan-sulfonic acid (HEPPS) buffer, barbital-acetate buffer, acetic acid-acetate buffer, carbonic acid-silicate buffer, 2-(N-morpholino)ethansulfonic acid (MES) buffer, carbonic acid-bicarbonate buffer, citric acid buffer or citrate buffer.

Photo-Latent Bases

In a preferred embodiment, a photo-latent base is used for increasing the pH value, whereby the photo-latent base releases the base upon being irradiated by the electromagnetic radiation, i.e. leading to an increase in the pH of the carrier medium (which preferably is a liquid). A photo-latent base of this type is advantageous in that, as before, no external base needs to be added to the system, but rather the (UV) light source that is used as an option according to the invention can initiate the shift in pH value from outside.

Examples of photo-latent bases include, e.g., α-aminoacetophenones, onium salts such as sulfonium or iodonium salts, as well as oxime sulfonic acid esters. Said compounds are known in the art and are described in numerous literature references.

Examples of photo-latent bases that can be used according to the invention are known, for example, from EP 0 898 202 A1, WO 94/28075 A1, WO 01/92362 A1, EP 0 970 085 A1, and WO 03/033500 A1, to which reference shall be made expressly herewith.

Well-suited photo-latent bases comprise N-substituted 4-(o-nitrophenyl) dihydropyridines, optionally substituted by alkyl ether and/or alkyl ester groups, as well as quaternary organic boron photoinitiators. Examples of N-substituted 4-(o-nitrophenyl)dihydropyridines include N-methyl-nifedipine, N-butyl-nifedipine, N-butyl 2,6-dimethyl 4-(2-nitrophenyl)1,4-dihydropyridine 3,5-dicarboxylic acid diethylester and a nifedipine according to the following formula:

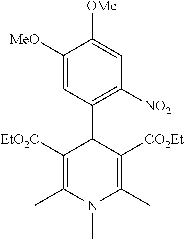 [SMILES: CCOC(=O)C1=C(C)N(C)C(C)=C(C(=O)OCC)C1c1cc(OC)c(OC)cc1[N+](=O)[O-]]

i.e., N-methyl-2,6-dimethyl 4-(4,5-dimethoxy-2-nitrophenyl)1,4-dihydropyridine 3,5-dicarboxylic acid diethylester. Examples of organo-boron compounds are disclosed in GB-A-2 307 473, such as for example

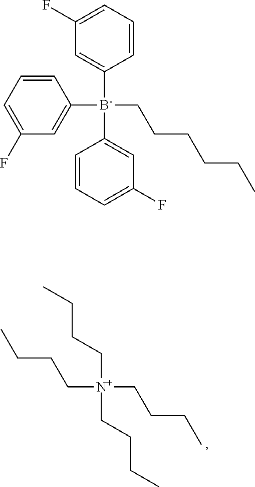

According to the prior art, in particular α-amino-acetophenone derivatives are known to be efficient photo-latent bases. Examples of α-amino-acetophenones that can be used in the process according to the invention include: 4-(methylthiobenzoyl)-1-methyl-1-morpholinoethane (Irgacure® 907ex, Ciba Spezialchemie) and (4-morpholinobenzoyl)-1-benzyl-1-dimethylaminopropane (Irgacure® 369ex, Ciba Spezialchemie), which are also disclosed in EPO 898 202 A1. An α-amino-acetophenone of the following formula is preferred:

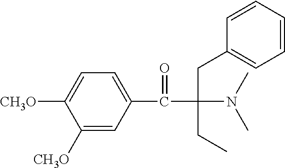

WO 94/28075 describes bases of the amine, ammonium or phosphane type that can be unblocked by UV. As blocking agents are used, in particular, alpha-ketocarboxylic acids, aromatic or N-heterocyclic formic acid, acetic acid or glyoxylic acid derivatives by means of which the bases can be converted to their non-reactive salts and which can be unblocked by irradiation. WO 97/31033 describes the photochemical release of bases with a pKₐ ˜12, N-benzyloxycarbonyltetramethylguanidine maybe mentioned here for exemplary purposes. Ionic salts of α-ammonium, α-iminium or α-amidinium ketones or alkenes that release the corresponding tertiary amine bases upon irradiation are disclosed, for example, in WO1998/38195 and WO 2000/10964. WO 1998/32756 discloses α-aminoketones that release amidine bases upon irradiation; corresponding α-aminoalkenes are disclosed in WO 1998/41524.

Examples of well-suited bases include, amongst others, tertiary amines and amidines, such as diazabicyclooctane, N-alkylmorpholines, tetramethylguanidine (TMG), diazabicyclononene (DBN), diazabicycloundecene (DBU) and imidazole.

Particularly well-suited amidines include photo-labile diazabicyclononanes, in particular 5-benzyl-1,5-diazabicyclo[4.3.0]nonane, whereby the 5-benzyl residue can just as well be singly or multiply substituted. Well-suited substituents on the 5-benzyl residue include, for example, halogen residues, such as chlorine or bromine, alkyl residues, such as methyl, ethyl or propyl, nitrile residues, nitro groups, alkoxy groups, such as methoxy or ethoxy, or aromatic residues condensated to the 5-benzyl residue, whereby, for example, a 5-(naphth-2-yl-methyl) residue or a 5-(anthracen-9-yl-methyl) residue can be derived from a 5-(benzyl) residue. Instead of the 5-benzyl residue, there may be, for example, a 5-(anthraquinone 2-yl-methyl) residue. Besides the possible substitutions on the 5-benzyl residue, the diazacyclononane residue may also be substituted further, such as, for example, in 5-benzyl-2-methyl-1,5-diazabicyclo[4.3.0]nonane. Besides the photo-labile diazabicyclononanes, it is also feasible to use the photo-labile diazabicycloundecanes, such as, for example, 8-benzyl-1,8-diazabicyclo[5.4.0]undecanes and the derivatives thereof. Analogous to the 5-benzyl residue of 5-benzyl-1,5-diazabicyclo[4.3.0]nonane, the 8-benzyl residue can be substituted further or can be replaced. Analogously, there is the possibility of further substitution on the diazabicyclononane residue.

In a special embodiment, the ascorbate antioxidant also acts as photo-latent base. As noted by the inventors, irradiation of an ascorbate solution with UV radiation, in particular with UV_{A} radiation, leads to an increase in the pH value.

It is also feasible to use photo-latent bases that contain two cleavable bases in one molecule. 1,4-Bis(1,5-diazabicyclo[4.3.0]nonanylmethypenzene is one representative of this kind of molecule. The synthesis of the photo-latent bases specified above is described, inter alia, in WO 03/033500 A1.

Pharmacological Agents

In an embodiment of the invention, and in particular in its embodiment as a liquid, gel or cream, the carrier medium contains one or more pharmacological agents. These can support the pharmacological effect of NO or act independent of NO in a therapeutically relevant manner considering the respective disease.

In one embodiment of the invention, and in particular in its embodiment as a liquid, gel or cream, the carrier medium contains one or more of the following pharmacological agents: anti-inflammatory agents such as, for example, non-steroidal anti-inflammatory drugs (NSAIDs) or corticoids, immunosuppressants, antibiotics, anticoagulants, anti-thrombotic agents, antiviral agents, antifungal agents, local anaesthetics, and analgesics.

Optional Addition of Another Antioxidant

In a preferred embodiment of the invention, at least one antioxidant is added while the pH value is being increased in step (d) or in a subsequent step (e).

In one embodiment, said at least one antioxidant corresponds to the at least one antioxidant provided in step (a) or (b). By this means, the antioxidant consumed during the generation of NO can be supplemented by new antioxidant.

Preferably, the at least one antioxidant that is newly added in step (d) or (e) is an antioxidant that can regenerate the previously added at least one antioxidant. Accordingly, it acts as an anti-oxidation synergist. Classically, the antioxidant is oxidised during the reduction of the corresponding substances. For the antioxidant to be regenerated, it needs to be converted to the reduced form by a stronger reducing agent (so-called “redox cycling”). As the first antioxidant to be reduced is known, the anti-oxidation synergist needs to have a more negative standard redox potential than same. Accordingly, for regeneration of ascorbate, which is used preferably and has a redox potential of +0.35 Volt, cysteine with a redox potential of −0.2 Volt (cysteine-cystine; 25° C., pH 7.0) is well-suited.

In a preferred embodiment, the antioxidant is an antioxidant from the substance class of the thiols. Preferred examples include: Cysteine, glutathione, N-acetylcysteine, dimercapto succinic acid, dimercapto propansulfonic acid, ethanthiol (ethylmercaptan), dithiothreitol (DTT), dithioerythritol (DTE), captopril, coenzym A, penicillamine, 1-propanthiol, 2-propanthiol, homocysteine, Mesna, methanthiol (methylmercaptan), and thiophenol.

In a particular embodiment, after addition of a thiol as antioxidant in step (d), the two components, i.e. NO donor, which preferably is nitrate, and thiol, are present at a molar ratio of 1:1-20. A preferred molar ratio in this context is 1:2-8, particularly preferably 1:3-7, and specifically a ratio of 1:5.

Photolytic Generation of NO

In a further embodiment of the invention, the carrier medium is irradiated with light for the purpose of photolytic decomposition of the NO and simultaneous production of NO in the process after step (d) or (e). A downstream photolytic generation of NO is advantageous in that, based on the physiologically relevant amount of NO that has already been generated, a decrease in the NO content (caused jointly by the continued reaction/decomposition of NO, and the release from the carrier medium) can be compensated elegantly by the photolytically-induced generation of more NO, in that no further addition of substances to the carrier medium is required, and in that the extent of the generation of NO can be controlled easily by means of the irradiation time and/or 2 0 irradiation intensity.

Light Source

According to the invention, a light source can be used in the process.

In the scope of the invention, a light source generates an electromagnetic radiation that contains the spectrum of the visible light, infrared light, and, in particular, the UV radiation. UV radiation comprises both UV_{A} and UV_{B} radiation in this context.

The type of irradiation of NO-generating starting substances is generally known to a person skilled in the art. It is feasible to use any electromagnetic radiation capable of degrading photo-labile NO derivatives while producing nitrogen monoxide. For example, in the scope of the present invention, nitrogen monoxide can be produced by means of photolytic cleavage using UV_{A} radiation with wavelengths of, for example, 320 to 400 nm. However, it is also feasible to use electromagnetic radiation of any other wavelength, which, alone or with the aid of chemical, physical or biological procedures, induces a photolytic cleavage of NO-generating NO precursors (NO derivatives).

The production of nitrogen monoxide can also take place in carrier media, and preferably in aqueous liquids, that are saturated with inert gases. The NO dissolved in said solutions that are saturated with inert gases (nitrogen (N₂), helium (H₂), argon, etc.) has a significantly longer half-life and can remain in solution even at higher concentrations. The maximum solubility of NO in aqueous solution is generally assumed to be approximately 2 mM. In this context, culture media or infusion media or infusion buffers shall also be understood to be aqueous solutions.

In a device for the implementation of the process according to the invention, the electromagnetic radiation can be emitted by a light source that can be attached outside and/or inside the device. It is important that the light exposure of the carrier medium including the reaction substances releasing the nitrogen monoxide is maximised with respect to the induced decomposition of the substance and/or release of nitrogen monoxide. The source of the electromagnetic radiation in this context can be an incandescent lamp or gas discharge lamp (low pressure- or high pressure-discharging) that is coated with corresponding fluorochromes, light-emitting diode (LED), organic light-emitting diode (OLED), LASER or or any other source of electromagnetic radiation capable of generating NO from the corresponding chemical precursors and/or substrates.

In order to optimally cleave the photo-labile NO precursors that are present in the carrier medium, the light source can emit electromagnetic radiation of wavelengths from 100 to 2,000 nm or electromagnetic radiation of any other wavelength, which, alone or with the aid of chemical, physical or biological procedures, can induce a cleavage of nitrogen monoxide precursors and thus can induce the production of nitrogen monoxide.

Accordingly, referring to photolytic cleavage, it is preferred to have the irradiation area of the device be made from a material that does not impair the properties of the energy of an electromagnetic radiation source that is required for optimum release of nitrogen monoxide or, due to its properties, generates the light properties required for light-induced release of nitrogen monoxide or optimises them or, in the case of pH-dependent generation of NO, promotes and optimises the pH-induced decomposition of nitrite.

The light used for irradiation of the photo-labile NO donor is in a wavelength range that depends on the respective NO donor. Accordingly, nitrites are irradiated, for photolysis with UV light, in a wavelength range between 320 and 400 nm, preferably between 340 and 380 nm, and particularly preferably at 365 nm. Referring to S-nitroso compounds, irradiation in the UV_{A} range (i.e. with wavelengths between 315 and 380 nm) is preferred, but light with a wavelength of up to 1,000 nm can also lead to a significant decomposition rate in this context.

Notably, the optimum wavelength for photolysis depends strongly on the metal cation. Especially in the presence of transition metal ions, e.g. Cu²⁺, aqueous nitrite solutions can absorb light of significantly longer wavelengths than is the case with “pure” nitrite solutions and therefore the nitrite ion can also be cleaved by light of wavelengths of 400-450 nm and even other wavelengths ≤450 nm while releasing NO. Likewise, S- and N-nitrosated chemical compounds can also be cleaved by photolysis with electromagnetic radiation ≤400 nm while releasing NO due to the relatively weak binding energy between NO and the residual molecule.

Device

According to a further aspect, the invention provides a device for implementation of the process according to the invention that is selected from the group containing bathing device, plaster, wound dressing, inhalator, oral irrigator, and spray.

In an embodiment in this context, the device comprises:
(a) An aqueous buffer system as carrier medium comprising a nitrite salt, ascorbate, and Trolox;
(b) a separate compartment comprising an acid;
(c) means for controlled release of the NO generated in the device; and
(d) optionally, an irradiation device for photolysis of NO donors.

Therapeutic or Cosmetic Use

Accordingly, in a special aspect, the invention provides a device for implementation of the process according to the invention that is suitable for use in the treatment or prevention of diseases, whereby the patient is being exposed to the NO released from the device.

Preferably, said treatment takes place by means of external or topical application. Accordingly, a skin area in need of therapy can be treated specifically by applying an NO-releasing plaster or a wound dressing to said area.

Alternatively, referring to a bathing device, the afflicted body part can be treated by immersion in the NO-containing liquid or by the NO-containing liquid being sprayed, poured on or poured over the body part.

Specifically, in this context, the process according to the invention can be used for stimulation of the metabolism of tissues through external application, in dermatology it can be used for treatment of surgical or accident-related wounds, chronic, non-healing and/or poorly healing and/or bacteria- and/or fungi-infested wounds as well as for the treatment of dermatological diseases from the realm of inflammatory, immunologically controlled and/or autoimmune diseases.

In a preferred embodiment, the disease treated with the device according to the invention is selected from the group containing neuropathic pain, varicose veins, ischaemia and thrombopathic diseases, allergies, skin infections, skin inflammations, atopic dermatitis, specifically neurodermatitis, dermatomyositis, and pemphigus vulgaris; wound defects such as chronic diabetic-neuropathic ulcus, venous ulcer, decubital wounds; primary healing wounds, secondary healing infected wounds, burn wounds, hidradenitis supparativa (acne inverse), warts, diaper rash, pseudofolliculitis barbae, skin graft complications, erectile dysfunction, angina pectoris, cardiac insufficiency, left-ventricular heart failure, coronary heart disease, pectanginous symptoms following myocardial infarction, anal fissure, spasms of the smooth oesophageal muscles, menstrual spasms, Reynaud syndrome, Buerger syndrome, peripheral arterial disease (PAD), peripheral vascular disease (PVD), inflammatory and autoimmune diseases of the skin (psoriasis, dermatitis, neurodermatitis), fungal diseases of the skin, bacterial, mycotic, and parasitic diseases of the skin (e.g. leishmaniosis), tinea cruris and tinea inguinalis.

Solutions prepared by means of the process according to the invention can preferably be used in the form of an inhalation spray for the treatment of obstructive pulmonary diseases. Moreover, they can be used for inducing a local vasodilation of narrowed or occluded blood vessels. In this context, it is preferred to directly apply the solution into the heart, for example by means of an endoscopic measure.

In one embodiment, local circulatory disorders in animals, such as laminitis in horses can be treated by means of the device according to the invention and, likewise, generally veterinary diseases that correspond to or resemble the human diseases listed above.

The device according to the invention can also be used for treatment of a muscular dystrophy (MD). Forms of MD that can be treated in this context comprise: MD-Duchenne, MD-Becker-Kiener, Emery-Dreifuss_MD-type 1, scapuloperoneal MD, reducing body myopathy (RBM), limb-girdle muscular dystrophy, congenital muscular dystrophy, distal muscle dystrophy, “vocal cord and pharyngeal weakness with distal myopathy” (VCPDM), myofibrillary myopathies, and myotonic dystrophies.

An inflammation that can be treated with the device according to the invention can be a bacterial, viral, fungal or parasitic infection. In this context, said bacterial infection can be caused, for example, by a bacterium selected from the group containing S. aureus, B. circulans, B. cereus, E. coli, P. vulgaris, P. acnes, S. pyogenes, S. enterica, V. anguillarum, K. pneumoniae, P. piscicida, P. aeruginosa, A. tumefaciens, M. tuberculosis, and M. ulcerans. Said fungal infection can be caused by a fungus selected from the group containing T. equinum, C. albicans, F. oxysporum, R. solani, B. cinerea, and A. flavus. The fungal infection to be treated can afflict the skin or nails in the form of an onychomycosis. Viral infections can be caused by one of the following virus families: Poxviridae, rotaviruses, papillomaviruses, parvoviruses, and varicella viruses. Preferably, the NO-releasing device can be used for treatment of skin infections, in which the Molluscum contagiosum virus is involved. Said parasitic infection can arise, for example, due to a parasite from the following genera: Plasmodium, leishmania, schistosoma, austrobilharzia, heterobilharzia, ornithobilharzia or cryptosporidium. The Plasmodium falciparum pathogen is to be noted specifically in this context.

In an embodiment, the device according to the invention can be used for treatment of the episodes of circulation problems that occur in cases of sickle-cell anaemia (sickle-cell crises). The hydroxyurea agent used in these cases is presumed to inhibit the formation of the deoxygenated T variant of the erythrocytes and thus to prevent the conversion to the sickle cell phenotype. If the released NO binds to haemoglobin, the R variant, which does not form sickle cells, is generated, which may be associated with an improvement of circulation and even the prevention of sickle-cell crises.

In another embodiment, the device according to the invention can be used for treatment of hair loss and, in this context, specifically of androgenic alopecia. The treatment includes both a reduction and termination of the hair loss and even the new growth of hair. Additional forms of hair loss that can be treated according to the invention comprise Alopecia praematura, Alopecia areata, Alopecia areata atrophicans, Alopecia totalis, Alopecia universalis, diffuse alopecia, Alopecia actinica, Alopecia mechanis such as Alopecia liminaris, Alopecia marginalis frontalis traumatica, Alopecia seborrhoica, Alopecia muciosa, and Alopecia parvimaculata. Analogous to the mechanism of effect of the drug minoxidil, the NO should be associated with increased circulation in the scalp and improved supply of blood, oxygen, and nutrients to the hair follicles.

According to the invention, the device can be used, for example, as follows:
1) on open wounds, since it has been evident, surprisingly, that the application according to the invention does not have a skin-irritating effect;
2) for MRSA prophylaxis in patients at risk; or
3) as a synergistic application in combination with conventional antibiotics, since it has been evident, surprisingly, that conventional antibiotics are capable of effectively controlling the residual inflammation as a consequence of the action of NO.

In a preferred embodiment, the device according to the invention is used for treatment of chronic lower limb wounds in diabetics. In this context, the treatment can act in terms of prophylaxis to reduce the risk of chronic wounds arising and to reduce the number of medical amputations. Accordingly, the reduction of the neuropathic leg pain and the establishment of an improved wound environment is associated with a significantly improved quality of life for the patient. Moreover, the reduced period of wound management is expected to lead to a significant reduction of the treatment cost.

Moreover, it may be possible to also address systemic diseases, such as, e.g., hypertension and related haemodynamic diseases, by treating extensive body areas.

In an embodiment of the invention, the device according to the invention is used for treatment of poorly healing wounds. Impaired arterial circulation and/or venous back-flow disorders are significant causes of the development and chronicity of lower limb wounds. NO-related arterial vasodilation improves the blood circulation in the afflicted tissue and the anti-thrombogenic effect of NO significantly promotes and/or facilitates venous back-flow of the blood. The NO-dependent improvement of both haemodynamic parameters is the crucial therapy-relevant aspect of a local or systemic effect that significantly reduces the risk of wound development and/or significantly accelerates wound healing. The NO supplied to the body parts to be treated by the device according to the invention can therefore be used successfully for therapy of poorly healing wounds.

In a particular embodiment, the device according to the invention is used for treatment of diabetic pain in the lower extremities, i.e. foot and/or leg. Diabetic pain is a very common event in the course of a diabetic disease. Diabetic foot/leg pain is the result of a persistent increase in the blood glucose concentrations, which is the underlying cause of the nerve and vascular damage observed during a diabetic disease. NO-related arterial vasodilation improves the blood circulation in the afflicted tissue and helps through an effect on the conduction of pain such as to reduce the pain. The NO supplied to the foot and/or leg from outside by the device according to the invention can therefore be used successfully for therapy of diabetic foot/leg pain.

In a special embodiment of the invention, the device according to the invention is used for treatment of patients with (skin) grafts and, in this context, in particular for treatment of poorly perfused flap grafts. The two haemodynamic parameters specified above, i.e. arterial circulation and venous back-flow, are also essential parameters for the therapeutic success of surgical flap grafts. Flap graphs are surgical techniques of plastic surgery, in which skin and/or tissue is transferred from one site (where it is non-essential) of an individual to another site. Usually, this concerns just skin flaps, but any tissue with or without skin as well as pedicled (i.e. including its respective blood-supplying vessels and nerves) as well as free (i.e. including connection of the blood vessels to the blood supply of the new environment) can be transplanted. The functional acceptance of the transplanted tissue depends exclusively on the arterial blood supply and controlled venous discharge. NO-related arterial vasodilation improves the blood circulation and therefore the needed supply of the flap graft and the anti-thrombogenic effect of NO promotes and facilitates venous back-flow of the blood. Therefore, NO preparations used from outside can assure and/or promote the success of a therapeutic option that is based on flap grafting.

In a further embodiment, the invention also provides a cosmetic process, in which the NO produced through the process according to the invention or the device according to the invention acts on the skin of a human.

Definitions

According to the invention, the term “treatment” shall be understood to mean any application of the device according to the invention to an individual that serves to mitigate or fully suppress the symptoms or causes of the disease or to impede, delay or postpone the progress of the disease.

In the context of the present invention, “prevention” shall be understood to mean preventing the manifestation of diseases and in particular of vascular or metabolic diseases, i.e. the reduction of their spread and the reduction of their impact on the morbidity and mortality of the population. The central strategy being to suppress or fully eliminate the causative factors of diseases.

In this context, prevention includes both primordial prevention, primary prevention, secondary prevention, tertiary prevention, and quaternary prevention.

The onset of primary prevention is before manifestation of the disease and it aims to prevent the new manifestation of a disease. Primary prevention addresses risk groups, healthy individuals, and persons without disease symptoms.

The onset of primordial prevention is even earlier, before primary prevention. It aims to prevent the manifestation of risk factors.

The onset of secondary prevention is at an early stage of the disease. It serves for early detection of diseases and the containment of their progress or of the disease turning chronic. The pathogenic process has already commenced, often in the absence of disease symptoms that could be perceived by the afflicted individuals. The target group are persons that participate in a prevention measure as healthy or symptom-free individuals, but become patients in the course of the diagnostic measure.

Tertiary prevention takes place after an acute treatment or the manifestation of a disease. It aims to prevent secondary damage and relapses. It addresses patients with chronic symptoms and rehabilitating patients. One pertinent example is the prevention of tumour relapses.

Moreover, there is quaternary prevention which aims to prevent unnecessary medicine or overdosing and takes into consideration the principle of «primum non nocere» as a basic pillar of all medicine.

The terms used in the patent claims, such as “comprise”, “include”, “contain” and the like, do not exclude further elements or steps. The use of the indefinite article does not exclude a plurality. A single device can perform the functions of multiple units and/or devices specified in the patent claims. Reference numbers as specified in the patent claims shall not be considered to limit the means and steps that are used.

The following embodiments are disclosed in line with the preceding description, and are part of the invention alone or in any combination thereof.

Embodiment 1 concerns a process for the production of nitrogen monoxide (NO) that comprises the following steps:
(a) Providing a carrier medium comprising at least one pH-labile NO donor;
(b) adjusting the pH value of the carrier medium to a pH value that induces the decomposition of the at least one pH-labile NO donor while producing NO;
(c) maintaining a pH value that induces the production of NO for a period of time that permits the production of a physiological amount of NO;
(d) increasing the pH value of the carrier medium;
(e) optional addition of another at least one antioxidant;
   whereby the carrier medium contains, in addition, at least one antioxidant in step (a) or the at least one antioxidant is added in step (b).

Embodiment 2: Process according to embodiment 1, characterised in that the carrier medium is selected from the group containing foam, gel, cream, and liquid, and preferably is an aqueous liquid.

Embodiment 3: Process according to embodiment 1 or 2, characterised in that the at least one pH-labile NO donor is selected from the group containing inorganic nitrite salt, alkyl nitrites such as isopentylnitrite, diazeniumdiolate derivatives, trans[RuCl([15]aneN4)NO]²⁺, 6-nitrobenzo[a]pyrrol, S-nitroso-glutathione, S-nitroso-thiol, S-nitroso-N-acetyl-D-penicillamine (SNAP), nitroaniline derivatives, 2-methyl-2-nitrosopropane, imidazolyl derivatives, nitrate esters, hydroxylnitrosamine, hydroxylamine, hydroxy urea, sodium nitroprusside, and preferably is an inorganic nitrite salt.

Embodiment 4: Process according to embodiment 1 to 3, characterised in that the at least one pH-labile NO donor is selected from the group LiNO₂, NaNO₂, KNO₂, RbNO₂, CsNO₂, FrNO₂, Be(NO₂)₂, Mg(NO₂)₂, Ca(NO₂)₂, Sr(NO₂)₂, Ba(NO₂)₂ or Ra(NO₂)₂, and combinations thereof, and preferably is NaNO₂.

Embodiment 5: Process according to any one of the embodiments 1 to 4, characterised in that the at least one antioxidant from step (a) and/or step (e) is selected from the group containing ascorbate and derivatives thereof, tocopherol, tocotrienol, tocomonoenol and derivatives thereof, Irganox®, Irgafose, butylhydroxyanisol (BHA), butylhydroxytoluene (BHT), glutathione, cysteine, thiolactic acid, alpha-lipoic acid, p-cumaric acid, ferulic acid, sinapinic acid, caffeic acid, gallic acid, protocatechuic acid, syringic acid, vanillic acid, polyphenolic compounds from the group of the anthocyanins, flavonoids or phytooestrogens, and preferably is a mixture of an ascorbic acid derivative and a tocopherol derivative.

Embodiment 6: Process according to any one of the embodiments 1 to 5, characterised in that the at least one antioxidant from step (a) is a mixture of ascorbate and (RS)-6-hydroxy-2,5,7,8-tetramethylchroman-2-carboxylic acid (Trolox) or a mixture of ascorbyl palmitate or ascorbyl stearate and alpha-tocopherol.

Embodiment 7: Process according to any one of the embodiments 1 to 6, characterised in that the carrier medium in step (b) contains nitrite, ascorbate, and Trolox at a molar ratio of 1:2-20:4-100, whereby the molar ratio: is nitrite<ascorbate<Trolox, and preferably the molar ratio is 1:5:10.

Embodiment 8: Process according to any one of the embodiments 1 to 7, characterised in that the pH value adjusted in step (b) is between 0.0 and 6.9, preferably between 2.0 and 6.0, particularly preferably between 5.0 and 6.0, and in particular 5.5.

Embodiment 9: Process according to any one of the embodiments 1 to 8, characterised in that the pH adjustment in step (b) takes place through the addition of an acid or through photolytic cleavage of a photo-latent acid producer.

Embodiment 10: Process according to any one of the embodiments 1 to 9, characterised in that step (c) takes a period of time of between 15 seconds and 60 minutes, preferably from 1 to 30 minutes, and particularly preferably of between 5 and 20 minutes.

Embodiment 11: Process according to any one of the embodiments 1 to 10, characterised in that the physiologically relevant amount of NO comprises an amount of between 0.05 to 1 mM and particularly preferably between 0.1 and 0.5 mM.

Embodiment 12: Process according to any one of the embodiments 1 to 11, characterised in that the pH increase in step (d) comprises one or more of the following properties:
a) is an increase of the pH value to pH 7 or more;
b) is an increase of the pH value by at least one full pH increment;
c) is an increase of the pH value to a pH value that is associated with a reduced generation of NO such that the amount of newly generated NO is equivalent to the decreasing amount of NO in the carrier medium.

Embodiment 13: Process according to any one of the embodiments 1 to 12, characterised in that the increase in pH in step (d) takes place through the use of a base or a basic buffer selected from the group containing NaOH, KOH, NH₄OH, Ca(OH)₂, NH₄OH, sodium hydrogen carbonate, phosphate buffer, barbital-acetate buffer, 4-(2-hydroxyethyl)-1-piperazinethanesulfonic acid (HEPES) buffer, tris(hydroxymethyl)-aminomethane (TRIS) buffer, 4-(2-hydroxyethyl)-piperazin-1-propansulfonic acid (HEPPS) buffer, barbital-acetate buffer, acetic acid-acetate buffer, carbonic acid-silicate buffer, 2-(N-morpholino)ethansulfonic acid (MES) buffer, carbonic acid-bicarbonate buffer, citric acid buffer or citrate buffer.

Embodiment 14: Process according to any one of the embodiments 1 to 13, characterised in that the pH increase in step (d) is to a pH that is between 7.0 and 12.0, preferably between 7.0 and 9.0, particularly preferably between 7.0 and 8.0, and in particular is 7.5.

Embodiment 15: Process according to any one of the embodiments 1 to 14, characterised in that the at least one antioxidant added in step (e) is selected from the group containing glutathione, cysteine, N-acetylcysteine, dimercapto succinic acid, dimercapto propansulfonic acid. Ethanthiol (ethylmercaptan), dithiothreitol (DTT), dithioerythritol (DTE), captopril, coenzym A, penicillamine, 1-propanthiol, 2-propanthiol, homocysteine, Mesna, methanthiol (methylmercaptan), and thiophenol.

Embodiment 16: Process according to any one of the embodiments 1 to 15, characterised in that, after step (d) or (e), the carrier medium in the process is irradiated with light for the purpose of photolytic decomposition of the NO and simultaneous production of NO.

Embodiment 17: Device for implementation of the process according to embodiments 1 to 16, characterised in that it is selected from the group containing bathing device, plaster, wound dressing, inhalator, spray, and oral irrigator.

Embodiment 18: Device for implementation of the process according to any one of the embodiments 1 to 17, comprising:
a) An aqueous buffer system as carrier medium comprising a nitrite salt, ascorbate and Trolox;
b) a separate compartment comprising an acid;
c) means for controlled release of the NO generated in the device;
d) optionally, an irradiation device for photolysis of NO donors.

Embodiment 19: Device according to embodiment 17 on 18 for use in the treatment or prevention of diseases, characterised in that the patient is being exposed to the NO released from the device.

Embodiment 20: Device according to embodiment 19, characterised in that the disease is selected from the group containing neuropathic pain, varicose veins, ischaemia and thrombopathic diseases, allergies, skin infections, skin inflammations, atopic dermatitis, specifically neurodermatitis, dermatomyositis, and pemphigus vulgaris; wound defects such as chronic diabetic-neuropathic ulcus, venous ulcer, decubital wounds; primary healing wounds, secondary healing infected wounds, skin graft complications, erectile dysfunction, hidradenitis supparativa (acne inverse), warts, diaper rash, pseudofolliculitis barbae, Reynaud syndrome, Buerger syndrome, peripheral arterial disease (PAD), peripheral vascular disease (PVD), inflammatory and autoimmune diseases of the skin (psoriasis, dermatitis, neurodermatitis), fungal diseases of the skin, bacterial, mycotic, and parasitic diseases of the skin (e.g. leishmaniosis), tinea cruris and tinea, inguinalis, muscular dystrophies, sickle cell anaemia, and alopecia.

Embodiment 21: Device according to embodiment 20, characterised in that it is used for treatment of chronic lower limb wounds of diabetics.

Embodiment 22: Cosmetic process comprising the action of NO on the skin of a human, characterised in that a process according to embodiment 1 to 16 or a device according to embodiment 17 or 18 is used.

### EXAMPLES

### Example 1

### One-Step pH-Induced NO Production Process

1.1 Materials:
Eco physics CLD 822: Quantification of NO
Reaction chamber: Quartz glass, approx. 100×100×10 mm (approx. 100 ml volume)
Buffer solution: 150 mM acetic acid, 150 mM NaOH in dist. water
Base: 1M NaOH
Sodium L-ascorbate
1M NaNO2

1.2 Experimental Procedure

A total of 0.56 g sodium L-ascorbate were dissolved in 98.6 ml buffer solution, transferred into the reaction chamber, and 1.4 ml NaNO₂ (1M) were added. Accordingly, the sodium nitrite concentration was 14 mM, the ascorbate concentration was 28.3 mM. The pH value of the final solution was measured to be 5.0.

For a period of 60 min, a 200 μl sample was taken in intervals of 2-3 minutes each and the NO content was quantified using the CLD system.

1.3 Results

The results of the NO measurements as a function of the reaction time are shown in FIG. 1. There is a continuous increase in the NO concentration, whereby a level corresponding to a concentration of 1.11 mM in the liquid was reached after 60 minutes.

### Example 2

### Two-Step pH-Induced NO Production Process

The aim of this experiment was to reach a therapeutically relevant final concentration for an extended period of time through an active change of the pH value.

2.1 Materials:

The same material as in Example 1 was used.

2.2 Experimental Procedure

Initially, analogous to experiment 1, 0.56 g sodium L-ascorbate were dissolved in 98.6 ml buffer solution, transferred into the reaction chamber, and 1.4 ml NaNO₂ (1M) were added. Accordingly, the sodium nitrite concentration was 14 mM, the ascorbate concentration was 28.3 mM. The pH value of the final solution was measured to be 5.0.

For a period of 45 min, a 200 μl sample was taken in intervals of 2-3 minutes each and the NO content was quantified using the CLD system.

2.3 Results

Initially, a continuous increase in the NO concentration was observed, whereby 1.5 ml NaOH (1M) were added in aliquots to the reaction chamber in the period of time between 10 and 15 minutes, which resulted in a final change of the pH value to a pH of 5.6. As a result, the NO concentration decreased initially and then levelled off from t=20 min at a level of 250+/−50 μM.

### FIGURE LEGENDS

The invention is illustrated in more detail based on the figures without limiting the invention to the figures shown. In the figures:

FIG. 1: shows the generation of NO by means of sodium nitrite as NO donor in an acetate buffer at pH 5.0 in the presence of ascorbate over a period of time of one hour (see Example 1).

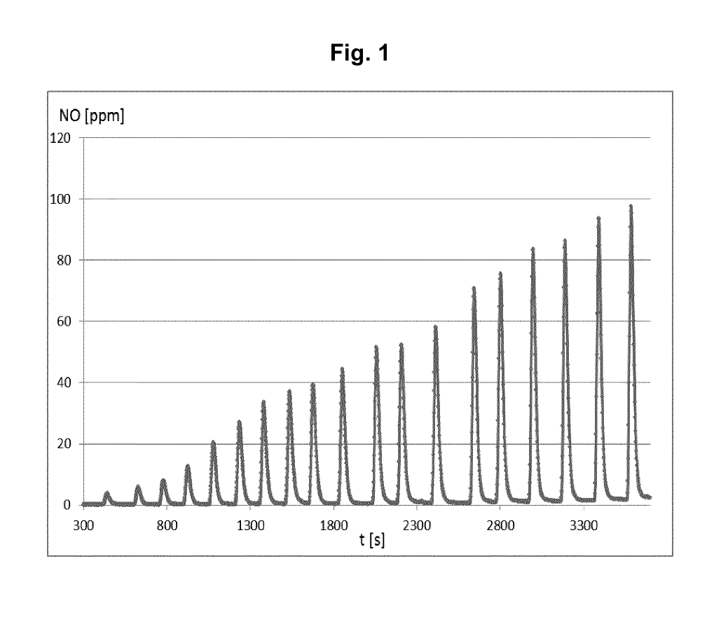

FIG. 2: shows the generation of NO according to the invention by means of sodium nitrite as NO donor in an acetate buffer in the presence of ascorbate with a first phase from t=0 to t=600 sec at pH 5.0, an increase in the pH value to pH 5.6 from t=600 to t=900, and a subsequent phase of NO generation at said pH of 5.6 (see Example 2).

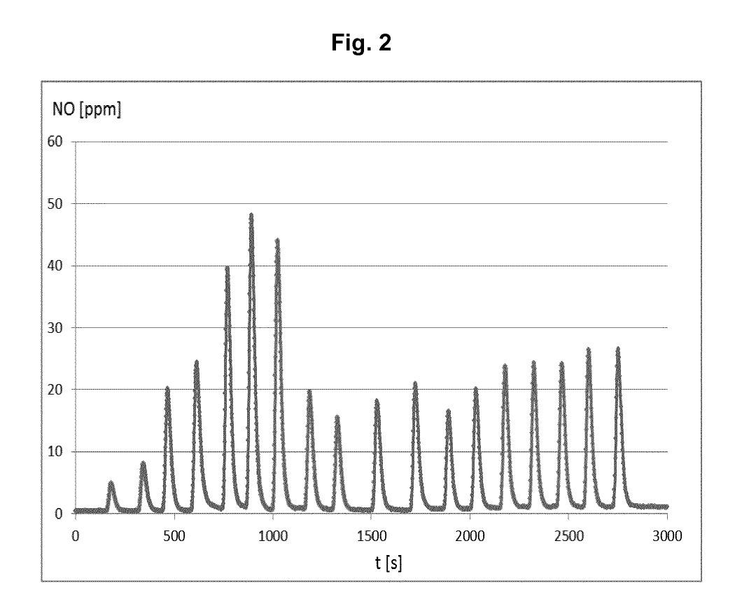

1. A process for the production of nitrogen monoxide (NO), comprising:
(a) providing a carrier medium comprising at least one pH-labile NO donor;
(b) adjusting a pH value of the carrier medium to a pH value that induces degradation of the at least one pH-labile NO donor while producing NO;
(c) maintaining the pH value that induces the production of NO for a period of time from 15 seconds to 60 minutes to permit production of an amount of 0.01 to 2 mM of NO; and
(d) increasing the pH value of the carrier medium by at least one pH level;
wherein at least one antioxidant is contained in the carrier medium in step (a) or at least one antioxidant is added in step (b), and
wherein the process optionally further comprises an addition of at least another antioxidant.

2. The process according to claim 1, wherein the carrier medium is selected from a group consisting of foam, gel, cream, and liquid.

3. The process according to claim 1, wherein the at least one pH-labile NO donor is selected from a group consisting of inorganic nitrite salts, alkyl nitrites, diazeniumdiolate derivatives, trans[RuCl([15]aneN4)NO]²⁺, 6-nitrobenzo[a]pyrrol, S-nitroso-glutathione, S-nitroso-thiol, S-nitroso-N-acetyl-D-penicillamine (SNAP), nitroaniline derivatives, 2-methyl-2-nitrosopropane, imidazolyl derivatives, nitrate esters, hydroxylnitrosamine, hydroxylamine, hydroxy urea, and sodium nitroprusside.

4. The process according to claims 1, wherein the at least one pH-labile NO donor is selected from a group consisting of LiNO₂, NaNO₂, KNO₂, RbNO₂, CsNO₂, FrNO₂, Be(NO₂)₂, Mg(NO₂)₂, Ca(NO₂)₂, Sr(NO₂)₂, Ba(NO₂)₂.

5. The process according to claim 1 , wherein the at least one antioxidant contained in the carrier medium in step (a) and/or the at least another antioxidant added is selected from a group consisting of ascorbate and derivatives thereof, tocopherol, tocotrienol, tocomonoenol and derivatives thereof, butylhydroxyanisol (BHA), butylhydroxytoluene (BHT), glutathione, cysteine, thiolactic acid, alpha-lipoic acid, p-cumaric acid, ferulic acid, sinapinic acid, caffeic acid, gallic acid, protocatechuic acid, syringic acid, vanillic acid, polyphenolic anthocyanin compounds, flavonoids, and phytooestrogens.

6. The process according to claim 1, wherein the at least one antioxidant is contained in the carrier medium in step (a) and is:
a mixture of ascorbate and (RS)-6-hydroxy-2,5,7,8-tetramethylchroman-2-carboxylic acid (Trolox), or
a mixture of alpha-tocopherol and at least one of ascorbyl palmitate and ascorbyl stearate.

7. The process according to claim 1, wherein the carrier medium in step (b) contains nitrite, ascorbate, and Trolox at a molar ratio of 1:2-20:4-100, and wherein the molar ratio: is nitrite<ascorbate<Trolox.

8. The process according to claim 1, wherein the pH value adjusted in step (b) is between 0.0 and 6.9.

9. The process according to claim 1, wherein the pH adjustment in step (b) is effectuated by an addition of an acid or by photolytic cleavage of a photo-latent acid producer.

10. The process according to claim 1, wherein the period of time in step (c) is between 1 and 30 minutes.

11. The process according to claim 1, wherein the amount of NO produced in step (c) is between 0.05 to 1 mM.

12. The process according to claim 1, wherein the pH increase in step (d):
(i) is an increase of the pH value to pH 7 or more; and/or
(ii) is an increase of the pH value to a pH value that is associated with a reduced generation of NO such that an amount of newly generated NO is equivalent to a decreasing amount of NO in the carrier medium.

13. The process according to claim 1, wherein the increase in pH in step (d) is effectuated by use of a base or a basic buffer selected from a group consisting of NaOH, KOH, NH₄OH, Ca(OH)₂, NH₄OH, sodium hydrogen carbonate, phosphate buffer, barbital-acetate buffer, 4-(2-hydroxyethyl)-1-piperazinethanesulfonic add (HEPES) buffer, tris(hydroxymethyl)-aminomethane (TRIS) buffer, 4-(2-hydroxyethyl)piperazin-1-propansulfonic acid (HEPPS) buffer, barbital-acetate buffer, acetic acid-acetate buffer, carbonic acid-silicate buffer, 2-(N-morpholino)ethansulfonic acid (MES) buffer, carbonic acid-bicarbonate buffer, citric acid buffer and citrate buffer.

14. The process according to claim 1, wherein the pH increase in step (d) is to a pH that is between 7.0 and 12.0.

15. The process according to claim 1, wherein the process further comprises addition of the at least another antioxidant, and wherein the at least another antioxidant is selected from a group consisting of glutathione, cysteine, N-acetylcysteine, dimercapto succinic acid, dimercapto propansulfonic acid, ethanthiol (ethylmercaptan), dithiothreitol (DTT), dithioerythritol (DTE), captopril, coenzym A, penicillamine, 1-propanthiol, 2-propanthiol, homocysteine, Mesna, methanthiol (methylmercaptan), and thiophenol.

16. The process according to claim 1, wherein the carrier medium is irradiated with light for photolytic degradation of the NO and simultaneous production of NO after step (d) or after addition of the at least another antioxidant.

17. A cosmetic process comprising contacting skin of a human with NO produced by a process according to claim 1.
